(19) United States
(12) Patent Application Publication
(10) Pub. No.: US 2023/0221248 A1
(43) Pub. Date: Jul. 13, 2023

(54) PROMPT VIRUSES INFECTION DETECTION USING THz SPECTROSCOPY IN A BREATHALYZER-LIKE CONFIGURATION

(71) Applicant: FLANIMUS LTD., Rehovot (IL)

(72) Inventors: Gabby SARUSI, Rehovot (IL); Rudrarup SENGUPTA, Beer-Sheva (IL)

(73) Assignee: FLANIMUS LTD., Rehovot (IL)

(21) Appl. No.: 17/916,010

(22) PCT Filed: Mar. 29, 2021
(86) PCT No.: PCT/IL2021/050354

§ 371 Date: Sep. 29, 2022

Related U.S. Application Data
(60) Provisional application No. 63/043,236, filed on Jun. 24, 2020.
(60) Provisional application No. 63/023,234, filed on May 11, 2020.
(60) Provisional application No. 63/005,329, filed on Apr. 5, 2020.
(60) Provisional application No. 63/001,338, filed on Mar. 29, 2020.

(51) Int. Cl. G01N 21/3581; G01N 21/84; G01N 33/497

(52) U.S. Cl. CPC G01N 21/3581; G01N 21/8483; G01N 33/497; G01N 2333/005

(57) ABSTRACT

A system for prompt virus infection carriers detection/screening using THz spectroscopy, which comprises a micro/nano-antennas array implemented as an antenna chip of predetermined shape and size, that has the maximum aspect ratio of the capacitor gap being sensitive to both P and S polarization, the array consisting of a plurality of printed micro-antenna elements, each of which having an equivalent inductor L of printed inductors and an equivalent capacitor C defined by gaps between printed contacts the length of the capacitor and the dielectric constant of a filler being between the printed contacts, to thereby determine a resonant frequency of the antenna element, the gaps are formed essentially along the cross diagonals of the each antenna element, thereby obtaining maximal aspect-ratio between the length of the capacitor and the gap width, that maximizes and sharpen the resonance effect of the each micro-antenna element; at least one capsule for holding the chip with the antennas array in a fixed position, preferably at the center, the at least one capsule being at least partially transparent to THz radiation range; means for applying material containing samples of viruses/exhaled biological ingredients to be detected that are exhaled into the gaps, for altering the dielectric constant of the filler and the resonance frequency; a THz spectrometer for scanning the samples and detecting shifts in the resonance frequency induced by the presence of the exhaled viruses/biological ingredients; at least one processor for processing the detected shifts in the resonance frequency and associating different shifts with different types of viruses/biological ingredients. The size of the array is matched to the beam size of the spectrometer, such that the entire radiation collimated beam will be captured by the antennas array, thereby maximizing the signal to noise ratio and the dynamic range.

### FIELD OF THE INVENTION

The present invention relates to the field of detection of viral disease infection. More particularly, the invention relates to a system and method for prompt viral infection detection in general, and COVID-19 (COronaVlrus Disease 2019) in particular, using THz spectroscopy.

### BACKGROUND OF THE INVENTION

The Corona virus among other respiratory viral related infections is a highly contagious virus. The Corona virus is spreading rapidly around the world, with symptoms of fever, cough, rash, red eyes breathing difficulty and in severe cases, also causes acute pneumonia that requires artificial respiration and death. It was found out that lethality is also related to over reaction of the immune system to viruses that generate what is known to be a “Cytokines' storm” in the lungs that basically cause rapid reduction of the Oxygen saturation level in the blood. Hundreds of thousands of patients worldwide have died from the corona virus infection during 2020.

Prompt and in a large scale detection of viruses in general, and the Corona virus in particular is considered to be one of the most important needs in order to control and eventually eliminate pandemics. Such capability enables to separate infected individuals from the healthy at the points of entry, as opposed to detection at the point of care, where the diagnosis of COVID-19 infected carriers is of an importance. This is traditionally done using biological based conventional methods such as Polymerase Chain Reaction (PCR), antibody and antigen blood tests.

The most commonly used methods to test an individual for virus's infection is PCR. It is a relatively accurate method but have a major disadvantage of being a very time consuming process (can take more than 6 hours) because of the nature of multiplication of the DNA count using multiple thermal cycles. Furthermore, it can take hours, and even days, to get the results from these test-kits due to a long queue and the cumbersome logistics. This time consuming process can be critical when dealing with mass tests of a large population, as in cases of wide spread pandemic.

Antibody blood tests is also problematic method in case of pandemic, since it can take test-kits 5-7 days after the initial infection to be detected, the time it takes for the human body to produce enough antibodies, required for detection. All in while, a human carrier continues spreading the virus and being contagious. Also, his/her medical condition can even get worse.

Park et al. (“Sensing viruses using terahertz nano-gap metamaterials”, BIOMEDICAL OPTICS EXPRESS 3551, Vol. 8, No. 8, 1 Aug. 2017) discloses a method for the detection of viruses using terahertz split-ring resonators with various capacitive gap widths. The size of the detected viruses ranged between 60 nm and 30 nm were detected by measuring the dielectric constants of the virus layers in the THz frequency range using metamaterial (nano-antennas array) based sensor chip, where each element contains an inductor and a capacitor having a nano-gap that was downsized to 200 nm in width. However, in order to be able to obtain substantial frequency shift required for detecting viruses, the method proposed by Park requires very high density of viruses inside a substance (about 10⁹-10¹⁰ viruses in a drop of 200 μL). Such extremely high density cannot be obtained by a sample taken from a patient either by swab nor with breathalyzer methods and therefore, so it is not practical for rapid testing. Also, it is challenging and more expensive to manufacture a narrow gaps using common lithography techniques. In most cases, there are residuals of photoresist materials that may fill the gap and deteriorate the measurement accuracy and more severely the uniformity between different arrays on the wafer. The probability that a substantial amount of viruses/biological fragments will enter such a narrow gap is lower.

In addition, the pattern of the metamaterial nanoantenna based sensors used by Park shown in FIG. 1 of Park responds only to one polarization of the THz field, which interacts with the sensor only if the transmitted field is perpendicular to the sensor's capacitor gap. However, in practice, collecting the samples from a patient is done when their accurate direction of arrival with respect to the sensor is essentially random. Therefore, Park's solution cannot be implemented efficiently.

It is therefore an object of the present invention to provide a device, system and method for fast detection of Corona (COVID-19) virus infection in particular and viral diseases in general, for preventing from a human carrier to continue spreading the virus and to enable the tested individual getting treatment as fast as possible after detection.

It is another object of the present invention to provide a device, system and method for fast detection of Corona (COVID-19) virus infection in particular and viral diseases in general using non-invasive breath test that provides almost instant results.

Other objects and advantages of the invention will become apparent as the description proceeds.

### SUMMARY OF THE INVENTION

A method for fast virus infection detection using THz spectroscopy, comprising:
a) providing a micro/nano-antennas array implemented as an antenna chip of predetermined shape and size, that has the maximum aspect ratio of the capacitor gap being sensitive to both P and S polarization, the array consisting of a plurality of printed micro-antenna elements, each of which having an equivalent inductor L of printed inductors and an equivalent capacitor C defined by gaps between printed contacts, the length of the capacitor and the dielectric constant of a filler being between the printed contacts, thereby determining a resonant frequency of the collective antenna elements in the array. The capacitor gaps are formed essentially along the cross diagonals of each antenna element, thereby obtaining maximal aspect-ratio between the length of the capacitor and the gap width, that maximizes and sharpen the resonance effect of the each micro-antenna element;
b) altering the dielectric constant of a filler by applying material containing samples of viruses/exhaled biological ingredients to be detected, into the gaps, thereby altering the resonance frequency;
c) detecting shifts in the resonance frequency induced by the presence of the viruses/biological ingredients that are exhaled in the gaps by scanning the samples using a spectrometer, such as a THz spectrometer; and
d) associating different shifts in the resonance frequency with different types of viruses/biological ingredients that are specific to a certain respiratory disease.

The size of the array is matched to the beam size of the spectrometer, such that the entire radiation collimated beam will be captured by the antennas array, thereby maximizing the signal to noise ratio and the dynamic range.

The metal surface of the antennas should be with high conductivity in order to obtain a high Q-factor of the antennas. In addition, the thickness of the metal surface should be thicker than the skin depth of the THz radiation in that particular metal. Such a careful design should provide high Q antennas array.

The method may further comprise associating corresponding shifts with healthy individuals and confirmed sick individuals, to further increase the probability of detection and minimize the false negative and false positive indications.

The method may further comprise applying machine learning analysis as an option after collecting large amount of THz spectra, and for wide frequency span, to further increasing the detection probability.

Scanning may be done using THz radiation in transmission or reflection mode spectrometers.

The combination of the inductance and the capacitance in the antenna elements may be in the range of pico-Henry and Femto-Farad, respectively so the resonance should be at the THz frequency range.

THz Spectroscopy may be done for dual polarizations P and S or single polarization spectrometer.

Spectroscopy may be done manually or by an automatic system that has a synchronous loader into the spectrometer, where the loader loads the tagged chips mounted inside the capsule (that is part of the breathalyzer like device) into the spectrometer, which frequency scans each chip for few ten of seconds. Then, a second algorithm analyzes resonance position and the frequency shift using effective known mathematical methods such as Gaussian Fitting, Weighted Mean etc. and provides accurate indications whether a person is not infected or infected.

The structure of each cell may be based on different capacitors that are in orthogonal orientations, to be responsive to different polarization P and S, in case the introduction orientation of the chip into the spectrometer is unknown.

The structure of each cell may be a diagonal type micro-antenna element (cell) called four arrowhead structure. In such a case, the length of the capacitor is maximized along the two diagonals of each antenna element, as well as the aspect ratio. The width of the capacitor can be varied from a relatively narrow gap, i.e., 200 nm and up to 3 μm. The tradeoffs are that in the small gap, the probability of capturing the nano particles inside the gap is smaller but the field enhancement is high. On the other hand, in a wider gap, the capturing probability is higher due to the larger area but the field enhancement is low. In most of the practical cases, it was found that a gap of 1-1.5 μm can be a good optimization.

The substrate may have a low doping level up to intrinsic semiconductor or insulator, thereby minimizing the free carrier absorption of the THz radiation, mainly in transmission mode.

Multiple tests may be performed by mounting a plurality of capsules inside a linear or circular magazine, which each time performs a predetermined linear/circular displacement, in order to advance a single capsule into the spectrometer, to coincide with the beam of the spectrometer.

Multiple tests may be performed by mounting a plurality of capsules inside a carousel like magazine with a plurality of capsules, which each time rotates by a predetermined angle, in order to advance a single capsule into the spectrometer, to coincide with the beam of the spectrometer.

The central axis of the carousel may be parallel to the beam, such that carousel is mounted horizontally and the capsule on it are amounted horizontally as well.

The carousel may be mounted horizontally, where the spectrometer is configured such that its beam will be vertically and so the capsules.

Preferably, the capsule that contains the chip is centered, to be co-aligned with the THz beam center.

A system for prompt virus infection carriers detection/screening using THz spectroscopy, which comprises:
a) a micro/nano-antennas array implemented as an antenna chip of predetermined shape and size, that has the maximum aspect ratio of the capacitor gap being sensitive to both P and S polarization, the array consisting of a plurality of printed micro-antenna elements, each of which having an equivalent inductor L of printed inductors and an equivalent capacitor C defined by gaps between printed contacts the length of the capacitor and the dielectric constant of a filler being between the printed contacts, to thereby determine a resonant frequency of the antenna element, the gaps are formed essentially along the cross diagonals of the each antenna element, thereby obtaining maximal aspect-ratio between the length of the capacitor and the gap width, that maximizes and sharpen the resonance effect of the each micro-antenna element;
b) at least one capsule for holding the chip with the antennas array in a fixed position, preferably at the center, the at least one capsule being at least partially transparent to THz radiation range;
c) means for applying material containing samples of viruses/exhaled biological ingredients to be detected that are exhaled into the gaps, for altering the dielectric constant of the filler and the resonance frequency;
d) a spectrometer, such as THz spectrometer, for scanning the samples and detecting shifts in the resonance frequency induced by the presence of the exhaled viruses/biological ingredients; and
e) at least one processor for processing the detected shifts in the resonance frequency and associating different shifts with different types of viruses/biological ingredients,
   wherein the size of the array is matched to the beam size of the spectrometer, such that the entire radiation collimated beam will be captured by the antennas array, thereby maximizing the signal to noise ratio and the dynamic range.

The capsule may be sealed to prevent contamination during a clinical trial.

The at least one processor may be further adapted to:
a) associate corresponding shifts with healthy individuals and confirmed sick individuals, to further increase the probability of detection and minimize the false negative and false positive indications.
b) apply machine learning analysis after collecting large amount of THz spectra and for wide frequency span, for further increasing the detection probability.

The system may further comprise a breathalyzer containing the antenna chip inserted inside a plastic capsule where the geometrical shape, plastic thickness and air gap are designed in accordance with the chip thickness and the nano-antennas characteristics, thereby minimizing the internal reflection and standing waves.

The system may further comprise a synchronous loader for loading tagged chips mounted inside the capsule into the spectrometer, which frequency scans each chip for few tens of seconds, analyzes the frequency shift and provides, during a diagnostic mode, accurate indications whether a person is infected or during a screening mode, whether a person is not infected.

The breathalyzer may further comprise a mouthpiece that is adapted to generate a fine mist after the breath test on the chip surface, for allowing the mist to dry out in few second after the breath test.

The capsule may be with no cap.

The system may further comprise a linear or circular magazine for performing multiple tests, inside which a plurality of capsules are mounted, the magazine each time performs a predetermined displacement, in order to advance a single capsule into the spectrometer, to coincide with the beam of the spectrometer.

The magazine may be a carousel, the central axis of which is parallel to the beam, such that carousel is mounted horizontally and the capsule on it are amounted horizontally, as well.

The carousel may be mounted horizontally, where the spectrometer is configured such that its beam will be vertically and so the capsules.

A breathalyzer containing the antenna chip inserted inside a capsule where the geometrical shape, thickness and air gap of the capsule are designed in accordance with the chip thickness and the nano-antennas characteristics, thereby minimizing the internal reflection and standing waves.

### BRIEF DESCRIPTION OF THE DRAWINGS

The above and other characteristics and advantages of the invention will be better understood through the following illustrative and non-limitative detailed description of preferred embodiments thereof, with reference to the appended drawings, wherein:

FIG. 1 (prior art) shows a simple schematic of a micro-antenna that contains two inductors in series with a capacitor, where the nano-particles are representing the viruses;

FIG. 2 shows a schematic configuration of a dual polarization sensitive micro-antenna, where the capacitor gaps have maximal aspect ratio by being positioned on the diagonals of each antenna element;

FIG. 3 shows a position of the viruses/biological fragments that the proposed device can sense;

FIG. 4 shows a part of the micro-antennas array;

FIG. 5A is a schematic illustration of a test configuration that includes a THz scanning spectrometer;

FIG. 5B is a schematic illustration of a vertical test configuration where the magazine is in the form of a carousel with a plurality of holes for holding a plurality of capsules;

FIG. 6 illustrates an embodiment of a breathalyzer configuration, in which the chip with the antennas array is inserted into a small cylindrical capsule that will become part of the breathalyzer;

FIG. 7 illustrates a loader for hosting several capsules, according to an embodiment of the invention;

FIGS. 8A-8B illustrate a mouthpiece mounted above the chip area, according to an embodiment of the invention;

FIG. 9 shows several capsules mechanically inserted into the THz spectrometer, using a magazine;

FIG. 10 shows the position of a tested capsule between the transmitter and the receiver of the spectrometer;

FIG. 11a illustrates a schematic structure of a novel cross-arrow LC resonant structure with Au as the deposited metal, where carbon QDs with varying concentration are placed in capacitive gap;

FIG. 11b illustrates a schematic structure of a novel cross-arrow LC resonant structure with Al as the deposited metal;

FIG. 12 is a microscopic image of fabricated arrowhead LC resonant structure, implemented in a chip substrate;

FIG. 13a illustrates the simulated natural resonant frequency and transmission spectra;

FIG. 13b shows the effect of 100 nm Carbon QD's (emulated as viruses) and the exhaled biological ingredients concentration on the shift in resonance frequency;

FIG. 13c shows the effect of Carbon QD (emulated as virus) concentration on the frequency shift ΔF;

FIG. 14. shows the sensitivity of Au, Al and TiN with increasing capacitive gap widths;

FIG. 16 shows varying resonance frequency for different capacitive widths (w);

FIG. 17a illustrates transmission data showing variation in resonance frequency with 100 nm Al deposited, for different capacitive gap widths;

FIG. 17b illustrates transmission data showing variation in resonance frequency with 200 nm Al deposited, for different capacitive gap widths;

FIGS. 18a-18f illustrate the transmission amplitude, showing the effect of C QD concentration resonance frequency for the structure for different capacitive widths;

FIG. 19 shows the simulation results with different QD sizes and concentration;

FIG. 20 shows plurality of micro-antenna structures are placed inside the plastic enclosure (capsule), with pre-determined distances of the chip from the front and back wall of the capsule;

FIG. 21 illustrates the S-parameter results from CST Studio suite; and

FIG. 22 Illustrates the effect of analyzing a raw spectrum from real measurement, using different algorithms.

### DETAILED DESCRIPTION OF THE INVENTION

The present invention provides a system and method for fast viruses' infection detection (such as Corona (COVID-19)) using THz spectroscopy, in order to prevent from the human carrier to continue spreading the virus and to enable the tested individual getting appropriate treatment as fast as possible.

The present invention provides a prompt method to scan many samples, taken from the suspected infected individuals, symptomatic or asymptomatic individuals from the entire population, such that the test results can be obtained in a very short time.

The method can be easily automatized, so it is expected that each sample scanning will take around 15 seconds only, so diagnosis will be received within less than 1 minute from the beginning of the breath test.

The test is based on principles from physics and electrical-engineering, rather biologic principles used by current biological based conventional methods. Accordingly, the viruses/biological fragments to be detected are treated as nano-particles, having a size (diameter) and a dielectric constant, just like any known nano-particle or quantum dots in physics or in material science that are measured using regular impedance spectroscopy to assess their dielectric constant. The DNA or RNA composition is not relevant for that kind of measurement, since the viruses/biological fragments are treated as nanoscopic material having a given size and a dielectric constant that is characterized by a very highly sensitive method, which is described herein.

Such nanoscopic particles detection is enabled by designing a miniature electronic circuit that following the implementation of viruses/biological fragments into the electronic circuit that will clearly affect its characteristics, such that even after introducing a very small amount of the nanoscopic particles the circuit characteristics will be altered with minimal ambiguity. It should be preferable to distinguish between nanoscopic materials with different size and different dielectric constant that basically represent different types of viruses. Moreover, it was found out that the amount of viruses that may enter into the capacitor gap is negligible, which raise the question what is actually measured during the breath test?

Several published papers about the content of viruses counts in each breath [XXX] show that their count can be as high as few thousands. Considering the fact that the area of the nano antennas on the chip is about 50% of the entire area and also that the area of the capacitors' gaps is about 2% of the antenna area, it seems that only 1% of the viruses may fall into the capacitor's gap. Therefore, each chip contains at most to 10 or 20 viruses. Such quantity of viruses can't be detected by the accumulative effect of about 15,000 nano-antennas that are implemented on each chip surface. Actually, a fixed virus is spread in a liquid drop having a density of 105 viruses per microliter and by using a high resolution, surface scanning microscope, it was possible to see that even in such a high density, the amount of viruses that are positioned inside the capacitor gap is very low (much less than 1%). This finding raised the question what really causes the frequency shift after the breath test of COVID-19 carrier as we observed during the clinical trials. The explanation is that unlike healthy individual breath that contains the standard ingredients, for respiratory related infected individual in general, and in COVID-19 carrier in particular, there are much more biological ingredients that are typical to each respiratory related disease. In case of COVID-19, it is well known that the viruses are multiplied inside the lunges cells and when they are breaking out after fracturing the cells, the lungs are full of cells debris, fat molecules, proteins, etc. In addition, in the initial phase after the infection, the immune system starts reacting by delivering different types of proteins (such as cytokines) that also cover the lungs' inner surface (in the severe cases what is known to be the Cytokines storm).

It is possible to automate of the test by automatic analysis of the resonance shift using different types of mathematical tools, mechanical automaton using tray and feeder that synchronously feed the tested chip position inside a transparent capsule that is a part of the breathalyzer-like device (i.e., a device that is capable of collecting samples of viruses/exhaled biological ingredients to be detected).

Different respiratory diseases may have different types of biological ingredients that are exhaled, namely with different dielectric constants and therefore, a resonance shift that is unique to every disease. The ambiguity of different diseases vs. different viruses loads can be explored by other diagnostic tools. When dealing with screening system, it is less important, since an individual can be denied from boarding an airplane in an airport scenario even if he has flu, pneumonia or COVID-19.

A COVID-19 carrier lunges contain biological ingredients that are completely different from the lunges of a healthy individual. Based on this analysis, it is assumed that that the observed frequency shifts during the COVID-19 breath test is related to all of these biological ingredients that are typical to COVID-19 carriers. It was assumed that this mixture of breath ingredients actually causes the shift. Although probably this “mixture” contains some viruses, as well, they do not play a key role in generation the frequency shifts when measured in the THz spectrometer. This is a main difference between known virus detection (presented in several papers such as Park et. al.) and the method of using a breath test, proposed by the present invention.

The proposed method uses a nano-antennas array (an “antenna chip”) having capacitor gaps along the cell's diagonal in order to detect the existence of the viruses/biological fragments on the antenna chip surface with a high signal to noise ratio. The principle is based on the known shift of the resonance frequency due to changes in the capacitance of the capacitor of the micro-antennas in the array, which are induced by the viruses/biological fragments adsorbed on its surface.

The present invention is based on the known principle which is built like miniature circuit that is made of two inductors in parallel with a single capacitor in series. Such circuit will have a resonance frequency (f res) that is given by the well-known relation:

fᵣₑₛ=½π\sqrt{(LC)},

where L is the equivalent inductance of the inductors and C is the capacitance of the capacitor in the circuit. In order to sense the existence of the viruses/biological ingredients (that do not exhibit any ferromagnetic properties, but only have dielectric properties), these viruses/biological ingredients should be brought into the capacitor gap so it will be an addition to the dielectric material (substrate and air) that already exists in that capacitor. This will change the capacitance of many of the capacitors within the nano-antenna array and hence, also the resonance frequency of each LC-antenna, as well as the collective average shift of the antennas array on the chip surface.

Each type of virus/biological ingredients having a different size and dielectric constant will have a different “signature” of that shift as explained above and therefore, can be detected with minimal ambiguity.

The main advantages proposed by the present invention are related to the unique architecture of the micro-antenna element, the unique architecture of the breathalyzer-like of device and to the method of conducting the breath test. As for the architecture, it is proposed to have and architecture where the size of the capacitor is maximized in the unit cell of each antenna as well as the aspect-ratio between the length of the capacitor and the gap width.

The second advantage is the method of conducting the test. The main problem with Swab test is that the amount of viruses is very small, so their direct effect of changing the nano-antenna characteristics is marginal to null. This is due to the fact that the few viruses collected with the swab are diluted in the buffer liquid and after collecting a small buffer liquid drop, the amount of viruses are negligible. On top of that, the viruses should enter the gap of the capacitor in order to make the frequency change. The relative area of the capacitor gap compare to the area of each antenna in the array is less than 1% so this further reduces the probability for viruses to do the change. However, if the test is done using a breathalyzer configuration (where the surface of the micro-antenna array is facing the opening of the mouthpiece) the spray of the breaths or coughs include biomaterials that are exhaling from the lungs that contains unique mixture of cells debris, cytokines and other proteins and some viruses. This mixture is unique to COVID 19 carriers and therefore, imposes a unique spectral shift being the signature of COVID-19 carrier. Therefore, the present invention allows the detection of artifacts that are created in the patient's lungs, which are typical to the damages caused by COVID-19. The amount of artifacts is much larger than the amount of viruses and therefore, the detection probability dramatically increases. It should be noted that it is expected that for each virus, there will be a signature of the spectral shift due to its effect on the lungs and respiratory system.

The clinical trials that have been conducted so far, showed a very high correlation between the frequency shift and the COVID-19 diagnosed individuals detected in parallel by PCR. This led to the conclusion that the THz spectral measurement actually includes the overall biological spices unique to the virus's activity within the lungs that is exhaled. Beside the actual viruses, this biological spices contain also cell fragments/debris, cytokines and other biological ingredients that are typically been found in the breath of corona virus's carriers in particular and in in other viruses infected individual's respiratory system in general. All these biological ingredients, when adsorbed on the micro-antenna array chip in general and in the capacitor gap in particular, provide a typical signature of frequency shift related to the ingredients exhaled due to each virus action inside the lungs and respiratory system. In addition, using machine learning tools after collecting large amount of THz spectra may farther increase the detection probability.

Measurement Method:

A reference chip without the viruses/biological ingredients should be measured in advance, so if all chips are uniform and have the same spectrum characteristics, each spectrum of the actual measurement is compared to the reference measurement. Otherwise, each chip should be measured separately as a reference that is stored, until the actual test measurement is done and then, to be compared to the reference. In order to analyze the spectral shift, the analysis of the spectral shift can be done manually or automatically using known mathematical tools, such as Gaussian fitting or Weighted average, so as to assess the spectral shift between the minima.

For the reference measurement and the test measurement, the chip with the antennas array may be mounted inside a cylindrical capsule made of polymer in a fixed position, preferably at the center. The capsule should be at least partially transparent to THz radiation range and sealed with a THz transparent cap to prevent viral external contamination during the spectral measurements and so is the lab. The spectral measurement can be in a class 1 laboratory Biosafety Level (Biosafety Level 1 is the level appropriate for work involving well-characterized agents not known to consistently cause disease in immune-competent adult humans and cause a minimal potential hazard to the laboratory personnel and the environment), without the need for biological hazard precautions means.

The main problem with detecting such frequency shift due to the existence of nanoscopic viruses/biological ingredients is that their effect is marginal in most of the cases and there is a need to design a proper nano-antenna architecture and to choose the right frequencies, such that their insertion in the capacitor gap will affect Δfᵣₑₛ and allow their detection. For viruses with known sizes span (20 nm-140 nm) and known dielectric constant, and for the biological fragments exhaling the resulting resonance frequency is within the Terahertz (THz) range, typically between 0.5 THz and 1.5 THz. The other biological ingredients that are associated with the effect of the viruses on the respiratory system such as cells fragments and cytokines are also with a typical sized and dielectric constant in average which further pronounce the typical frequency shift signature of each virus respiratory infection.

Therefore, a planar antenna with the correct architecture can detect the existence of viruses and the biological species inside the gap of the capacitor, due to the change in the capacitance, and the resulting change with respect to fᵣₑₛ.

Such frequency scanning of the antennas array can be done by commercially available THz spectrometers (which show the intensity of THz radiation as a function of wavelength or of frequency. Since each element leaves its spectral signature in the pattern of lines observed, a spectral analysis can reveal the composition of the object being analyzed). In order to achieve such resonance frequency, the combination of the inductance and the capacitance in that LC antenna should be in the range of pico-Henry and femto-Farad, respectively. Only by using such low inductance and low capacitance the effect of viruses' and fragments insertion to the capacitor gap can be detected. Calculating such combination yields an inductor with one to two loops having a diameter around of 10 μm-40 μm and a capacitor with a plates area of about 1 μm² to 8 μm². The gap occupied by the dielectric material of such capacitor should be in the order of 100 nm to around 3 μm. Such structure was already investigated [Ref. 1] for direct detection of viruses mounted in a very high concentration in the lab, where the shift off Δfᵣₑₛ of such antenna is in the order of few tens of GHz. In that particular reference the viruses (bacteriophages) concentration was extremely high, causing that shift. In a real day to day testing (using swabs or breathalyzers) such high concentration of viruses can't be reached. As mentioned, the effect of the viruses on the respiratory system causes that the exhaled biological ingredients has a unique signature (in term of a resonance shift) that can be detected using the resonance micro-antennas array on the chip surface.

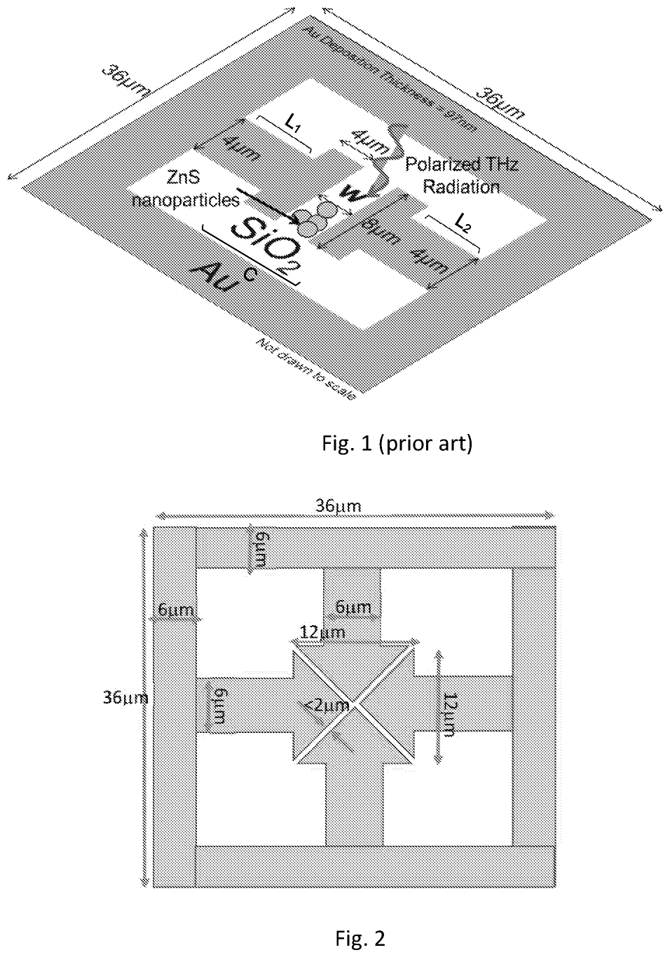

FIG. 1 (prior art from Park et al.) schematically illustrates a micro-antenna that contains two inductors L1 and L2 in parallel, with a single capacitor C in series, as shown in Ref. 1, where the nano-particles are representing the viruses.

The present invention architectures presented in FIGS. 2 to 4 provide a high efficiency THz absorption device having a larger capacitor gap area and aspect ratio as well as responsivity for dual polarizations P (parallel to the plane of incidence) and S (perpendicular to the plane of incidence). Dual polarizations are beneficial, since even if the spectrometer has circular polarization or even when working with one polarization spectrometer still the other polarization exists but with lower magnitude—i.e., residual polarization and therefore, it is possible to use both of them and save signal power. In addition, if the residual polarization is not exploited and not detected by the device, it contributes to unwanted noise. In addition, even with a single polarization spectrometer, any orientation of the nano-antenna capacitor can be aligned with the spectrometer polarization. Therefore, a circular polarization sensitive device architecture (as proposed by the present invention) is preferred.

The aspect ratio of the capacitor in the structure promise a high signal to noise ratio. Such structures which are not-sensitive to polarization enable also an easy insertion into a mass scan spectrometer without the need for specific orientation of the antenna array.

The present invention further provides an automatic system that has a synchronous loader and a spectrometer, where the loader loads the tagged chips (Bar code or RFID etc.) into the spectrometer, which scans each chip for few seconds analyze the frequency shift and provides a relatively accurate indication whether a person is infected or not infected, as described below.

The system will be able to increase the probability of adsorbing the viruses/biological fragments on a larger area of the capacitors' gap within the frame of the inductor, and at the same time will be able to respond to both THz radiation polarization P and S of the THz spectrometer. It should be mentioned that even with spectrometers that are considered to be linearly polarized, there is always residual radiation on the other polarization. If the antenna is design to pick up both polarizations the signal will be larger and the other residual polarization will not induce noise. In addition, the arbitrary orientation of the chip with respect to the spectrometer polarization can be overcome by a device that is responsive to both polarizations, as proposed by the present invention. The structure is based on different capacitors that are in orthogonal orientations along the diagonals of the square antenna unit cell. Therefore, they can be responsive to different polarization P and S, where the reference geometry is identical with respect to the two polarizations. Such examples of different devices architectures are presented in FIGS. 2-4 and are called “four arrow-heads-like” configurations.

FIG. 2 shows a schematic configuration of the dual polarization sensitive sensor, using four arrows-head like forms, where the capacitor gaps have maximal aspect ratio by being positioned on the diagonals of each antenna element. This four arrows head configuration provides a large aspect ratio, which is capable of capturing more viruses due to the fact that the gap is longer. Also, a large aspect ratio causes the applied electromagnetic field to be enhanced and located inside the capacitor. Another advantage of a large aspect ratio is the ability to increase the gap and thereby, also the chance to capture more viruses. A larger gap also decreases the variance between different micro-antenna cells and contributes to the uniformity of the array.

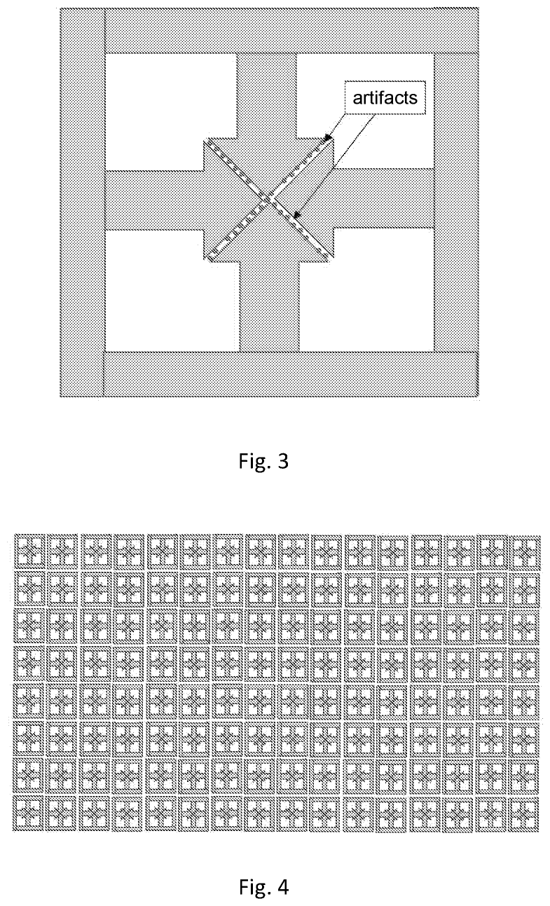

FIG. 3 shows a position of the viruses/biological ingredients that the device can sense, using the configuration of FIG. 2.

FIG. 4 shows a part of the micro-antenna cells array, where each cell has the configuration of FIG. 2.

Simulations and Analysis

LC Resonant Metamaterial Micro-Antenna Structure Simulation

The simulations for a single unit LC resonant micro antenna have been carried out by finite element solver COMSOL Multiphysics (a cross-platform finite element analysis, COMSOL, Inc., Burlington, Mass. U.S.A.) with both time domain and frequency domain electromagnetic solver, with accurate frequency steps. 100 nm carbon Quantum Dots (QD's) have been used to emulate COVID-19 viruses. The approximation rendered appreciable consistency in determining resonance frequency shift at different virus concentrations. All simulations and further fabrications are done on Si substrate with Au/Al deposition, to minimize extra processing steps and ease mass-production. Finally, the transmission amplitude through the resonating structure has been simulated. The minima of the transmission amplitude signify the resonating frequency.

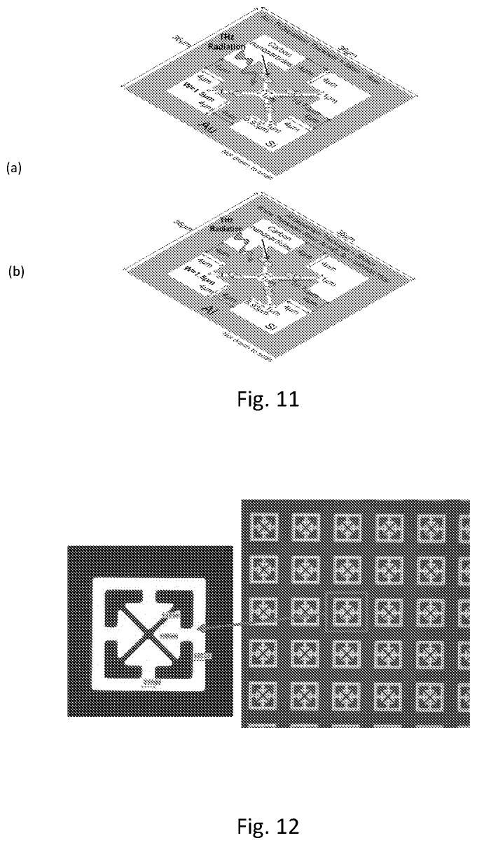

FIG. 11a illustrates a schematic structure of a novel cross-arrow LC resonant structure with Au as the deposited metal, where carbon QDs with varying concentration are placed in capacitive gap. FIG. 11b illustrates a schematic structure of a novel cross-arrow LC resonant structure with Al as the deposited metal. This arrangement helped simulating COVID-19 like detection, as well as the sensitivity of the metamaterial device to the virus. The novel cross-arrowhead structure (which is physically independent of the P and S polarization of the incident electromagnetic wave) proposed by the present invention, can be placed in any orientation in the spectrometer making the detection system more robust and simpler. Moreover, this structure is much more sensitive to smaller changes in the dielectric media, due to larger capacitive areas, which is extremely beneficial, as the probability of virus getting attached in between the arrowhead plates increases manifold. This makes the proposed structure more efficient for virus detection.

FIG. 12 is a microscopic image of fabricated arrowhead LC resonant structure, implemented in a chip substrate.

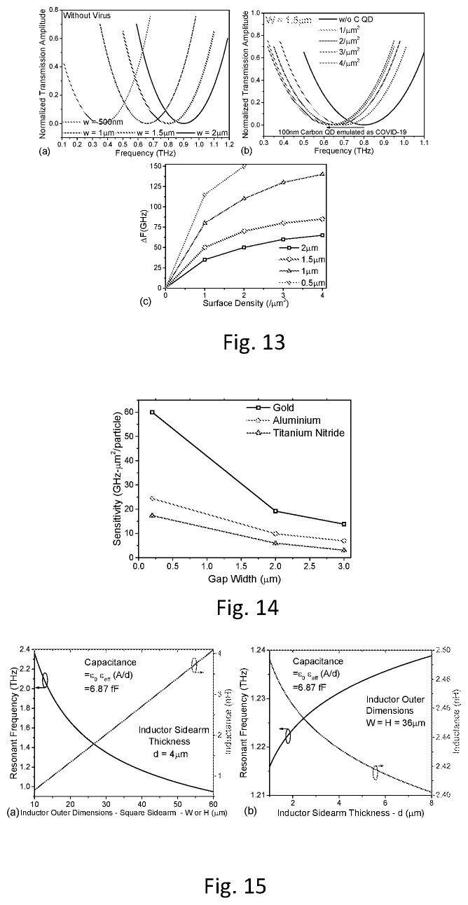

FIG. 13a illustrates the simulated natural resonant frequency and transmission spectra, depicting natural resonant frequency with various capacitive widths with various capacitive widths. A capacitive width of 1.5 μm have been used for the simulation. All testing kits were made with the above arrowhead chip with w=1.5 μm.

FIG. 13b shows the effect of 100 nm Carbon QD's (emulated as viruses) and the exhaled biological ingredients concentration on the shift in resonance frequency.

FIG. 13c shows the effect of Carbon QD (emulated as virus) concentration on the frequency shift ΔF.

The Effect of the Type of Metal Used

It is known that metals with different metallic properties will give slightly varied resonance frequencies, given that relative permittivity of all the metals is more-or less the same. However, some metals are more sensitive to changes in electric fields, and in this case, the change in the electric field is caused by changing the dielectric media. The expected change in dielectric constant by the addition of viruses/biological ingredients will render equivalent change in capacitance in Femto-Farad range. Hence, the resonating frequencies will be bound to the THz range for micron-dimensioned structures. In such small structures with nanoscale change in electrical parameters, sensitivity is of a major concern to the design of LC resonant structures.

Sensitivities of Au, Al and TiN are shown with increasing capacitive gap widths in FIG. 14. Interestingly, Au has the highest sensitivity, followed by Al and then TiN.

The Effect of Geometry

The structure and its related physics of the LC resonant metamaterial structure are entirely dependent on its geometry. As per the resonance formula,

$${f}_{0} = 1 / ( 2 π \sqrt{L C} ) , C = k {ε}_{0} ( \frac{A}{w} ) ,$$

designing the structure depends on the following structural properties,
1. Inductor outer dimensions (W and H),
2. Inductor sidearm thickness (d),
3. Capacitor area or the area of the metallic plate acting as the capacitor (A),
4. Distance between the capacitor plates (w).

FIGS. 15a and 15b show the dependency of the inductor dimensions of the resonance frequency of the structure. FIG. 15a shows a graphical representation of the dependency of the inductor outer dimensions and FIG. 15b shows a graphical representation of the dependency of the inductor sidearm thickness on resonant frequency of the structure.

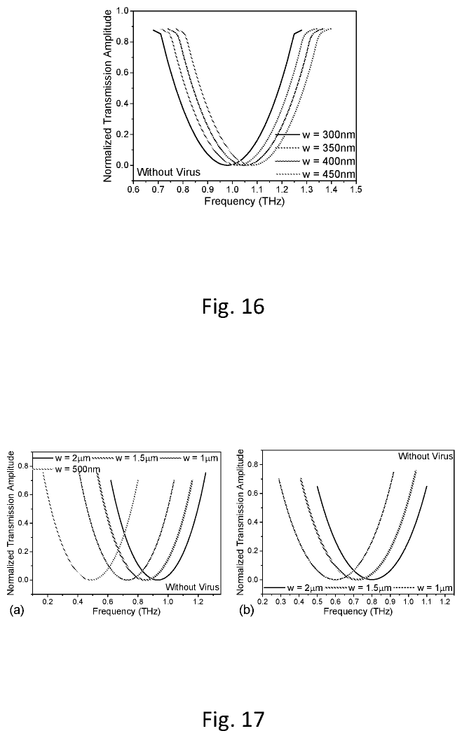

Similarly, if the capacitive width is varied, there will be a substantial variation in resonance frequencies. FIG. 16 shows varying resonance frequency for different capacitive widths (w), where the resonance frequency is characterized by the dip in the transmission spectrum.

Effect of Metal Deposition Thickness

It is imperative that any change in the metal deposition thickness will change the effective capacitive area (A) of the micro-antenna structure. Therefore, the change in capacitance will change the resonance frequency of the LC resonant structure. Quantifiably, if the metal deposition thickness is increased, the effective capacitance will increase, thereby reducing the resonance frequency, as shown in FIGS. 17a-17b.

FIG. 17a illustrates transmission data showing variation in resonance frequency with 100 nm Al deposited, for different capacitive gap widths. FIG. 17b illustrates transmission data showing variation in resonance frequency with 200 nm Al deposited, for different capacitive gap widths.

The Effect of Virus/Biological Ingredients Concentration (Load)

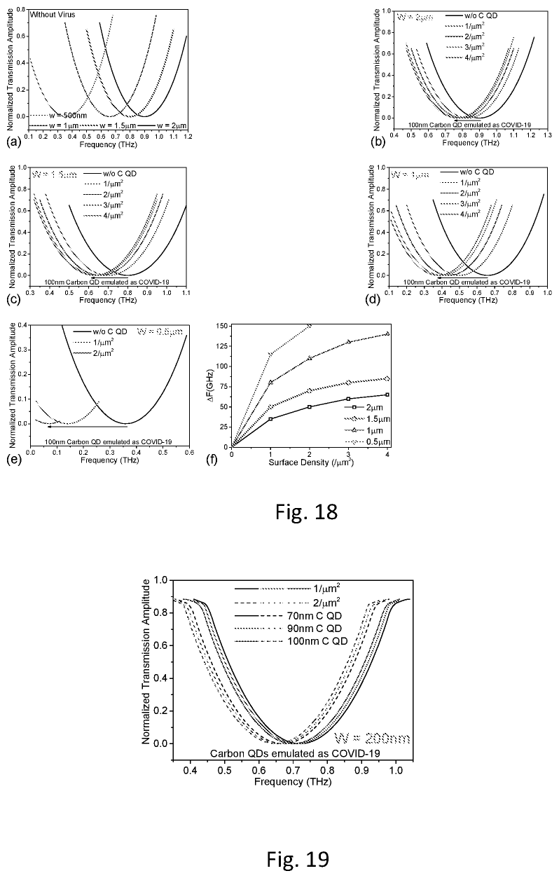

The structure proposed by the present invention is definitely responsive to different virus concentration, since viruses/biological ingredients of different concentrations in the capacitive width have different effective dielectric constant and hence, there will be variation in resonance frequency with virus load. The resonance frequency change seems to saturate with increasing virus concentration, as shown in FIGS. 18a-18f.

FIGS. 18a-18f illustrate the transmission amplitude, showing the effect of C QD concentration resonance frequency for the structure for different capacitive widths: without any inserted C QDs that emulate viruses (FIG. 18a); for w=2 μm (FIG. 18b), for w=1.5 μm (FIG. 18c), for w=1 μm (FIG. 18d), for w=0.5 μm (FIG. 18e) and a combined graph showing the saturating resonance frequency with CQD concentration at different capacitive widths (FIG. 18f).

Specificity to Detect a Particular Virus

It is known that if the dielectric media is changed by altering the virus/biological ingredients shape or size, the effective dielectric constant will always be different any will give different resonance frequency. Each different virus/biological ingredients will therefore give its unique resonance frequency signature, which can be used to recognize that virus/biological ingredients. In the simulations, carbon quantum dots of varying sizes have been used to emulate the effect of different viruses/biological ingredients in the LC resonant metamaterial chip.

FIG. 19 shows simulation results of the effect of different QD sizes and concentrations on the resonance frequency. It can be seen that there is a definite difference between resonance frequency of each size of the QD. Hence, the structure proposed by the present invention is definitely sensitive to geometry of the materials falling in its capacitive region. Moreover, the structure is seemingly more sensitive to minute changes in the dielectric media compared to the linear structure. This is because S. J. Park, et. al. reported that the linear structure becomes increasingly insensitive to viruses of ^{˜}100 nm dimension. This is an advantage since coronavirus dimensions are reportedly 100 nm-140 nm.

LC resonant Chip and Capsule (THz Scanning System) Simulation

Simulation of the entire THz scanning system for coronavirus detection comprises simulating an array of LC resonant structures, placed inside the breathalyzer enclosure. A plastic enclosure (capsule) has been designed, which is semi-transparent to THz radiations for our fabricated chips. Simulations are done in CST studio suite (a high-performance 3D EM analysis software package for designing, analyzing and optimizing electromagnetic components and systems, SIMULIA Solutions, Hertogenbosch, the Netherlands), which emulated the entire THz scanning system. CST simulations are done to avoid creation of standing waves due to the capsule walls and the thickness of the Si wafer that antennas array in fabricated on. The micro-antenna chip container capsule has a fixed distance between its two walls surrounding the chip. Simulations and designs are made in such a way that the standing wave creation is minimized at the resonance frequency of the chip. CST simulations is also used to minimize the ‘Fabry-Perot oscillations’, due to the Si chip. Si wafer of a particular thickness creates ‘Fabry-Perot oscillations’ due to multiple refractions and reflections of light from its walls. Simulations also minimize this effect by designing a geometric specific cavity inside the capsule container which houses the micro-antenna chip. The capsule that contains the chip is centered, to be co-aligned with the THz beam center.

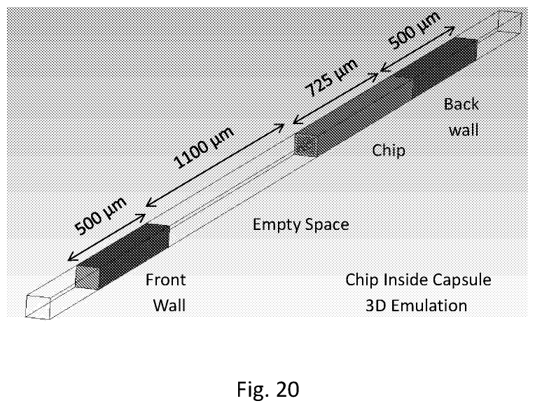

A plurality of micro-antenna structures fabricated on the chip are placed inside the plastic enclosure (capsule), with pre-determined distances of the chip from the front and back wall of the capsule, as shown in FIG. 20. This helped simulating the Fabry-Perot oscillations due to multiple reflections and refractions from the walls of the Si chip. Among multiple oscillating regions, a singular frequency dip has been identified, which signify the resonance frequency of the antenna.

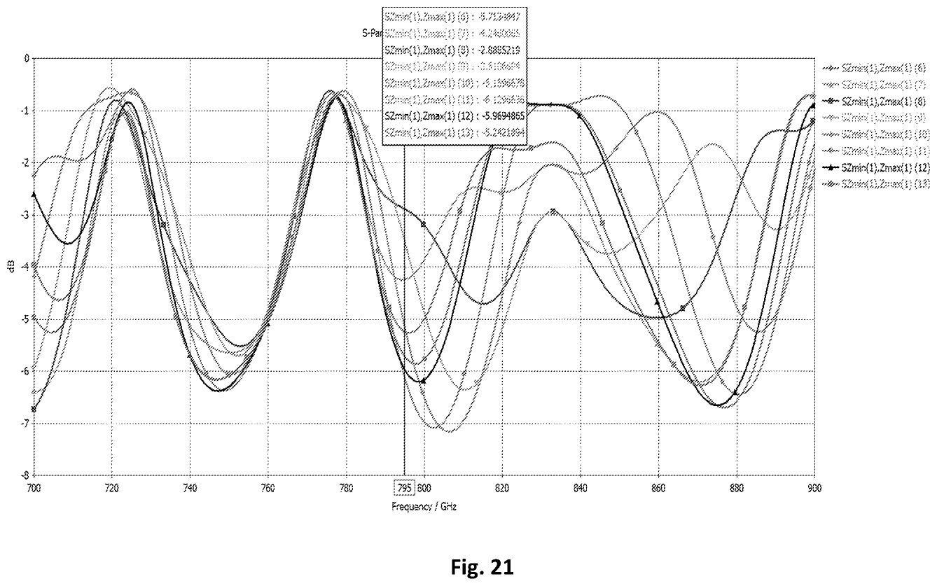

For example, in order to obtain a correct position of the Si chip inside the capsule, the air gap between the chip and the front wall of the capsule has been varied in steps of 30 μm, from the original distance of 1100 μm. The change in S_{Z(min)}, S_{Z(max)}-parameters has been recorded. It can be seen that there is a change/shift in the response but there is no clear null. Also, it was found that the periodicity is of about 180 μm. The S-parameter results from CST Studio suite are illustrated in FIG. 21.

FIG. 20. 3D Emulation of chip Inside capsule with proper distances and thicknesses of each layers for accurate simulation results.

FIG. 21. S_{Z(min)}, S_{Z(max)}-parameters recorded with varying the empty space region between the chip and the front wall of the capsule. The Fabry-Perot oscillations due to the entire Si chip and capsule-system is visible.

Breathalyzer Airflow Simulation

The breathalyzer kit consists of a plastic capsule which contains the LC resonant metamaterial chip and a separate blower, so that the tested individual can blow on the chip directly. Proper air flow design inside the blower ensures that the particles exiting the tested individuals mouth gradually settles on the chip, instead of flying away or stick to the breathalyzer walls. The main idea is to have a mouthpiece that is narrow at the top where it interfaces with the mouth and become broader at the chip level. In that architecture, there will be a minimal amount of large droplet of saliva but rather, fine mist that will dry out during the time period from taking the mouthpiece off and putting the cap on.

Automation and Analysis of Coronavirus-THz Spectrum

During the clinical trials, many noisy spectra have been encountered. Some spectra have a broadened resonance region, with noise in the resonance dip itself. Some samples have a wavy nature due to the Fabry-Perot oscillations between the metamaterial chip and the capsule. Most prominently, inconsistent resonance frequency has been experienced over chips fabricated on the same mask design. The chip-to-chip variation of resonance frequency ranges up to 70 GHz, making it almost impossible to decide on a programming routine to Gaussian fit within the same frequency range. In order to overcome these problem, an automation program has been implemented according to the following steps:

The Concept of Automated ΔF Extraction:
1. Broad range of frequency spectrum approximately around the resonance region is selected for further analysis.
2. Total number of files to be analyzed are taken from the user, accordingly, a while loop is designed.
3. A Gaussian function is used Gaussian fitting of the spectrum separately for the reference and measurement spectra.
4. ΔF array stores the difference between the minima of the Gaussian fitted measurement and reference. For Improper Gaussian fitting, the user is alerted to re-check that particular sample.

Proper Gaussian fitting around the resonance region is essential for correct determination of ΔF. A correctly weighted gaussian fit will perfectly approximate the resonance region and give the optimized resonance frequency.

Fine-Tuning of the Algorithm

Interestingly, all standard smoothing techniques like b-spline, gaussian, etc., uses average method to find out the smoothed curve. This feature of every smoothing technique appears to be the major constrain to the used samples, because in the resonance region where photocurrent value gets really low at the resonance frequency, there are less data points to work with. In the broader is the resonance-range where the spectrum gets a bit noisy, the greater is the number of data points. Therefore, the average concept will undermine the actual precise resonance region with lesser data points, and shift its weight to the parts of the resonance region which have more data points.

The concept of calculating the local minima of the spectrum at resonance region, and apply Gaussian fitting, and/or calculate the resonance frequency by weighted mean of the local minima points has been introduced. Therefore, our spectrum is processed in 3 ways:
1. The spectrum around the resonance region is Gaussian fitted.
2. Local minima points of the spectrum in the resonance region is calculated and the spectrum with only the minima points are Gaussian fitted.
3. The local minima points of the spectrum in the resonance region is calculated and the resonance frequency is calculated by weighted mean, with more weight being given to the minima with the least value.
4. The Hilbert envelope of the raw spectrum is also intended to analyze the resonance frequency shift.

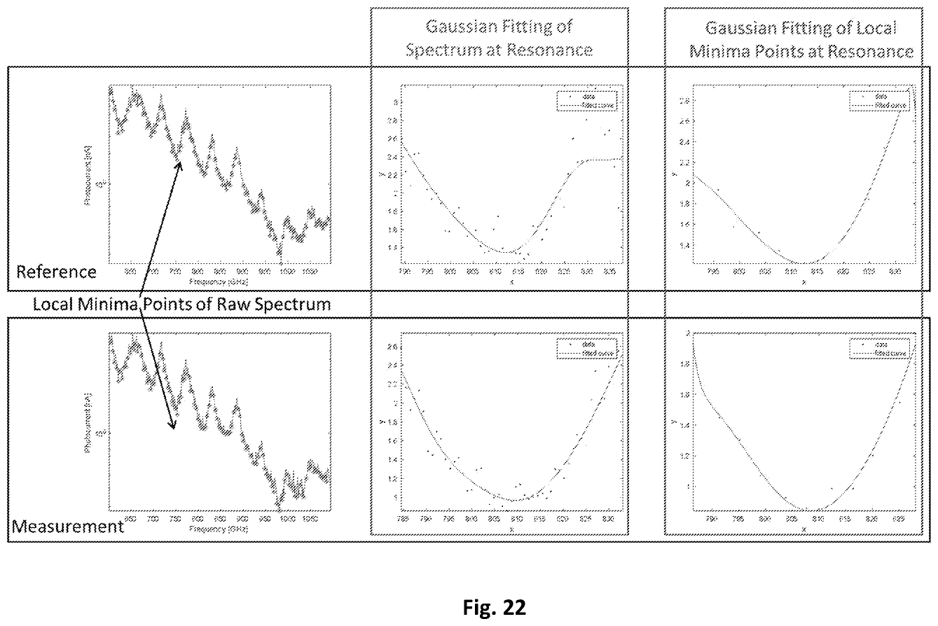

FIG. 22 Illustrates the effect of analyzing a raw spectrum from real measurement, using different algorithms.

An algorithm is present on the top of these three procedures, which selects the best ΔF suitable for the spectrum.

For fast virus's infection detection tests the process from taking the breath sample from the individual until receiving the final result should be short in term of seconds or to the most 1 minute. The present invention proposes the breathalyzer method to collect the samples tests from individuals using the proposed micro-antennas array chip.

Breathalyzer configuration—in this configuration, the chip with the antenna array is put in small compartments inside the breathalyzer, about 4 cm down from the entry point. The tested individual blows or coughs several times into the inlet of the mouthpiece. The design of the breathalyzer is done in a way that the biological fragments and viruses hit the surface of the chip and adsorbed on it while the exhale air can be ventilated out through side openings. The chip is mounted inside a capsule that composes of the lower part of the breathalyzer. After taking the mouthpiece off a cap is covering the chip surface. The entire capsule is then positioned inside the THz spectrometer that scans it over the designated frequencies range. The entire breathalyzer is disposable and the breathalyzer's materials especially the capsule should be made of materials that are relatively transparent to the THz radiation and are FDA approved.

The capsule design (size, thickness and air gaps) is also adapted to optimally match the impedance of the chip and the spectrometer's transmitter, so as to eliminate the generation of unwanted reflections and standing waves (which add ripple to the measured spectrum) such design can be done using different types of antenna design software, such as CST Studio Suite etc.

Mass Measurements of Chips

The present invention uses a THz spectrometer system that can automatically scan the relevant expected absorption wavelength, that is synchronized with a loader that feeds the chips with the viruses/biological ingredients being tested. A reference clean chip is measured in advance and each spectrum is compared with the that reference chip, in order to analyze the spectral shift, due to the existence viruses/biological ingredients inside the antenna's capacitor gap.

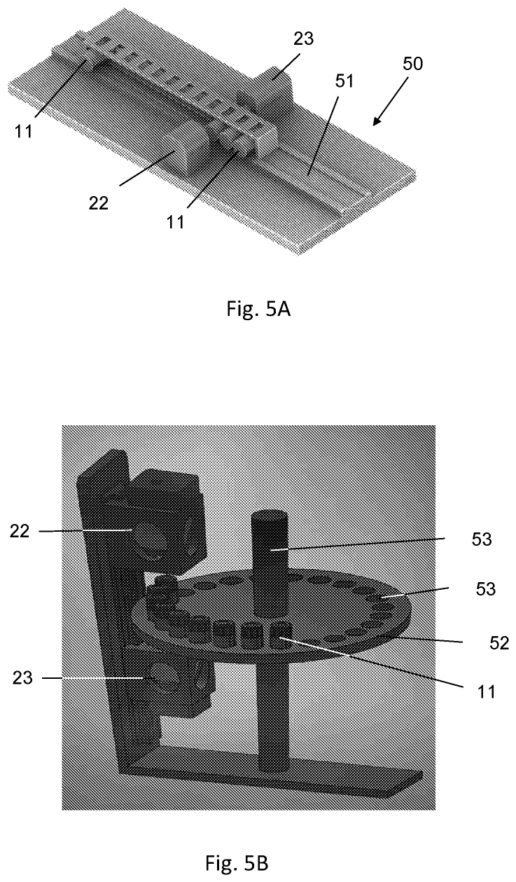

FIG. 5A is a schematic illustration of a test configuration that includes a THz scanning spectrometer, consisting of a transmitter part 22 and a receiver part 23 and a linear loader 50, which feeds chips with the antennas array to be scanned into the gap between the transmitter part 22 and a receiver part 23.

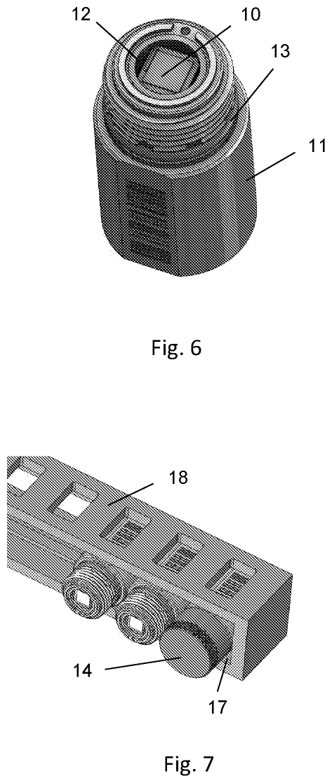

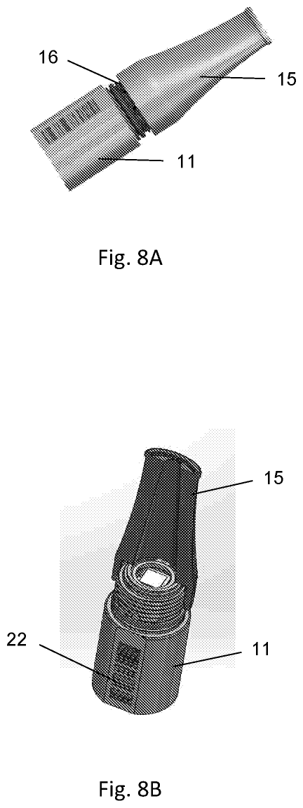

Each chip is mounted in a small cylindrical capsule 11, where loader 50 holds a plurality of capsules in a magazine 18 (in this example, a linear magazine) that holds all capsules in a row, such that the chip area in each capsule 11 will be perpendicular, centered and co-aligned with respect to the beam center of the THz scanning spectrometer. Loader 50 comprises a conveyer 51 for advancing one capsule each time, to be scanned. Scanning is synchronized with the loader that feed the chips with the viruses/biological fragments, to be tested. The a detailed description of capsule 11 is specified with respect to FIGS. 6, 8A and 8B below.

Alternatively, the magazine may be in the form of a carousel 52 with a plurality of holes 53 for holding a plurality of capsules 18, as shown in FIG. 5B. According to one embodiment, the central axis of the carousel may be parallel to the beam, such that carousel is mounted vertically, which the spectrometer will be configured such that its beam will be vertical.

Carousel 52 is held in a horizontal orientation by a vertical axis 54, which is connected to a motor (not shown) that rotates the carousel 52 upon receiving a command from a controller (not shown). In this arrangement, the transmitter part 22 and a receiver part 23 are mounted by a holder 55 in a vertical position, such that the center beam of the THz scanning spectrometer will be vertical and parallel to the axis 54. The location of axis 54 is determined such that the edge of carousel 52 will be inside the scanning gap between of the transmitter part 22 and a receiver part 23 and the controller will be able to locate the center of each hole, holding a capsule 11, to be aligned with the scanning center beam.

Each time, Carousel 52 rotates by a predetermined angle determined by the controller, in order to advance a single capsule 11 into the center of the scanning area of the THz spectrometer, to coincide with the beam center of the THz spectrometer.

The physical principles of the infected viruses' detection is described below. A bio sample that is collected from a human individual is attached to a special low cost chip, made for example of silicon, glass, silica or plastics (such as PET). The chip design enables precise and accurate results. The chip size may be for example, 8×8 mm, where the active central area may be smaller than the total chip area, for example, 7×7 mm. The active central area contains few tens by few tens of micrometer size antennas array, made of printed electrodes only, so the active central area of 7×7 mm contains about 15,000-20,000 elements. Each element is a passive circuit of one to two microscopic inductors in parallel with a capacitor in series forming a nano-antenna with a specific resonance. The capacitor is located in the center of the two inductor loops, as previously shown in FIGS. 2-4 above. The size of the overall array (i.e., 7×7 mm) should co-align with the spectrometer beam diameter, such that the beam will overlap with the entire antennas array area.

It is possible control the resonance frequency of the device by designing its geometry and dimensions. For example, a device that has an external dimension of 36×36 μm and a capacitor gap of 2 μm will have a resonance frequency around 0.85 THZ, if it is made using gold metal on Si wafer. Changing of the metal types or the wafer may change the absorption resonance frequency.

A variety of metals such as Al, Au, Al-ALD and substrates such as quartz and glass may be also used.

The detection of the viruses/biological fragments is done by activating the array that contains approximately 20,000 elements and can be covered with the viruses/biological ingredients using a terahertz spectrometer radiation that scans over chosen frequencies range.

Once the Corona viruses/biological ingredients are adsorbed on the chip active area with in the capacitor gap, there is a change the capacitance and the resonance frequency of the cell. Since it is known that influenza viruses in general and the Corona family in particular can induce resonance frequency shift at the THz range, the inserted viruses/biological ingredients will drastically alter the capacitance, and thus the resonance frequency of the cell will be shifted from the original reference measurement. This shift will always be red shift since adding the biological fragments to the capacitor's gap replacing the air will always increase the capacitance and reduce the resonance frequency.

The shift between the resonance peak with and with-out the viruses/biological fragments can reach a value of around 200 GHz depending on the antenna dimension, materials etc. Putting this chip with the approximately 20,000 elements inside the beam of a THz spectrometer and illuminating it on the surface side so the beam covers the entire antennas, absorption of the THz radiation will occur at around the resonance frequency. This shift is estimated to be around 3-50 GHz, which depends on the micro-antenna shape, size, metal and wafer types as well as the virus/biological fragments types exhaled from the respiratory system. Therefore, it is possible to conclude what kind of viruses are on the tested chip. The sensitivity of the system depends from the amount of elements that are covered by the THz beam, the aspect ratio of the capacitor gap, the size of the capacitor within the antenna dimension, the gap between the capacitor plates, the material of the substrate, the metal of the antennas and may also the absorption enhancement, due to the plasmonic effect in narrow capacitor's gap.

Signal processing tools such as machine learning can also be applied to the overall spectrum obtained from the individuals, in order to reduce false positive and false negative indications.

In one aspect, the present invention is directed to a method to use a breathalyzer like test method along with a mass test system to be able to test an individual with in approximately 15 to 60 seconds time frame.

FIG. 6 illustrates an embodiment of a breathalyzer configuration, in which the chip 10 with the antennas array is inserted into a small cylindrical capsule 11 that will become part of the breathalyzer. The chip 10 is mounted in a fixed position with respect to the capsule outer interface, preferably at the center. The capsule's materials are as much as transparent as possible to THz radiation range. The capsule is made of cylindrical compartment 12 for the chip and a cap that closes the top surface using a threaded cap 14 (shown in FIG. 7), that can be sealed with an O-ring 13. The reason for taking these precautions is due to the fact that viruses' contamination can penetrate out of the chip compartment during the test. This arrangement safely closes the cap 14 with O-ring 13 and minimizes the outside contamination. The design of the internal capsule that hold the chip is done in a way that takes into account the chip materials and thickness and the capsule material and thickness allows the terahertz beam propagation. In addition, a careful design should be made with the geometry of the capsule with respect to the thickness and the material of the chip to matched them in order to prevent standing waves and ripples in the spectrum that is been collected. This is crucial for the proper and less noisy spectral collection.

In a breathalyzer configuration, the cap 14 is removed from the top of the capsule and a mouthpiece 15 is then mounted instead (as shown in FIGS. 8A-8B. The mouthpiece 15 includes an opening 16 at the bottom, close to the chip plane so the air that is blown on the chip can be ventilated out of mouthpiece 15 after the breath biological contents have been stick to the chip surface, such that the air that ventilated out of mouthpiece 15 is almost clean, while all the artifacts are trapped on the chip surface. The individual blows several times or coughs several times into the mouthpiece 15 inlet. After blowing, the mouthpiece 15 is removed and the capsule 11 is sealed with the cap 14 that presses the O-ring 13 to obtain total sealing. The capsule 11 is then mechanically inserted into a compartment 17 between the transmitter and the receiver of the THz spectrometer, introducing a transmission test (or reflection test). The capsule 11 with the chip 10 is positioned in a very precise position so the measurement will have the same parameters.

Methods to Identify Infected Individuals from Un-Infected Individuals

During the tests which are taken from individuals, fraud attempts may occur, mainly by people that want to hide their sickness by mimicking breathing or coughing into the breathalyzer mouthpiece.

This problem may be overcome by each or combination of four modalities:

1. The mouthpiece can be provided with a CO₂ sensitive sticker that should change its color upon sensing the CO₂ when real breathing or coughing is made.
2. A 8-12 microns thermal camera can be positioned in front of the individual, to identify the hot breath streams coming out from the nuzzles at the bottom of the mouthpiece.
3. The mouthpiece can be made in a form of whistle that has a specific sound when the blowing in done properly.
4. Using a small balloon that expand to a certain volume when blown properly

The above measurements can be also used to quantify the proper breath strength that is needed for optimal biological ingredient collection.

Knowing the exact resonance frequency of the antennas array, the THz spectrometer system can automatically scan only the relevant expected absorption frequencies ranges upon a start signal from an external command and control system. Such scanning is synchronized with a loader that feeds the chips with the breath sample viruses. A reference chip (one chip as a reference for all measurements or each chip is a reference) without the viruses/biological ingredients is measured in advance and each spectrum is compared with that reference chip, in order to analyze the spectral shift. Each spectrum is compared with the reference chip, in order to analyze the spectral shift due to the viruses/biological ingredients inside the antenna's capacitor gap. A positive or negative indication regarding a specific virus infection can be determined by the shift induced by the breath sample, compared to the reference sample (in case that the processing of the chip is not repeatable, a one by one reference may be needed).

(a high-performance 3D EM analysis software package for designing, analyzing and optimizing electromagnetic (EM) components and systems, SIMULIA Solutions, Hertogenbosch, the Netherlands).

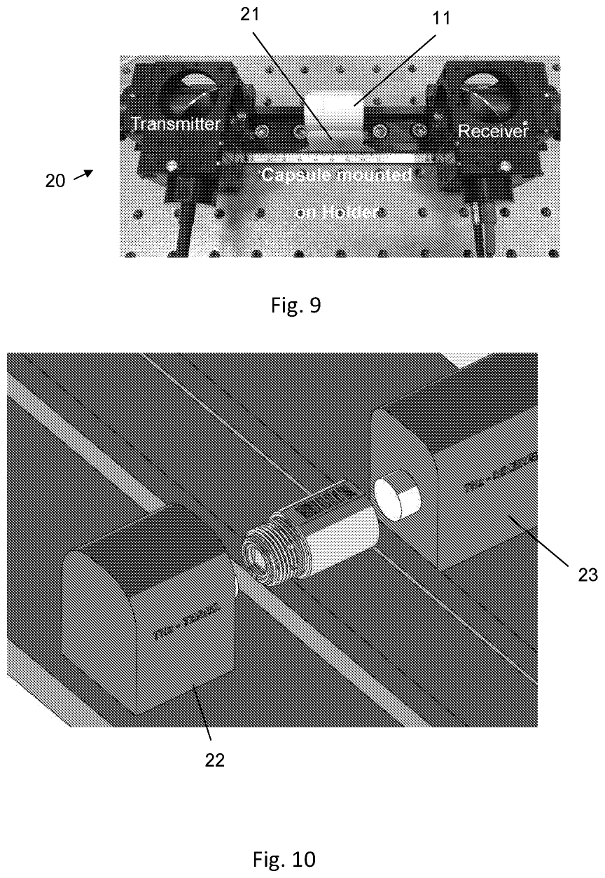

Prompt viruses' detection is essential for mass testing of the capsules with the chips. For that purpose, right after taking the breath samples and sealing the capsule, the capsule is 11 mechanically inserted into an analyzer system 20, containing the THz spectrometer and a holder 21, as shown in FIG. 9. The insertion into the analyzer system 20 can be done one by one (as illustrated in FIG. 9), or using a magazine of several capsules 11.

The magazine may be a linear (or circular) magazine 18 (illustrated in FIG. 7 above) with several capsules, which each time performs a predetermined linear displacement, in order to advance a single capsule into the analyzer system 20, to coincide with the beam of the spectrometer.

A top command and control software module with a GUI is used to communicate with the spectrometer and the with capsule 11 that is inserted to the right position inside magazine. Once the capsule 11 is in the correct position, a start scan command is given to the spectrometer. Upon finishing the measurement, the spectrometer sends a finish signal to the command and control system, so data collection will start. The collected data will be analyzed locally or remotely in a computational cloud.

Each collected spectrum is associated to a capsule serial number. The number of the capsule can be read by bar code 22 or by RFID device that can be inserted as part of the capsule. Each capsule ID is then associated with an individual ID. Upon obtaining a positive result (that the individual is infected), the individual may get a message to his/her cell phone or to any other massaging means. In such case, it is recommended to do a second test in order to verify the result using the same method or by any other method. In case of confirmed positive result, the authorities are informed and the infected individual will get instructions from the authorities regarding what measures should be taken. Negative textual messages may also be sent to the individual or to the authorities that will allow the individual a green pass for a certain period of time after the test (such as to connection flights etc.).

According to another embodiment, the analyzer system 20 may comprise high power UV LEDs that upon detecting that a sample in a capsule is tested with positive indication (i.e., that the sample contains viruses), a control circuit will activate the UV LEDs to apply high power UV radiation for sterilizing the capsule. Alternatively, any capsule to be tested in the spectrometer will be sterilized using UV radiation. Any test that resulted with a positive indication will be repeated, for verification.

After completing the test, any capsule that provided a negative indication will be aggregated in a regular trash container. Any capsule that provided a positive indication will be aggregated in a special trash container which will be labeled as biologic contaminated trash, to be treated carefully until final evacuation. Alternatively, all capsules will be considered positive and treated as biological waste.

FIG. 10 shows the position of a tested capsule between the transmitter 22 and the receiver 23 of the spectrometer.

### REFERENCES

1. Vol. 8, No. 8|1 Aug. 2017|BIOMEDICAL OPTICS EXPRESS 3551: Sensing viruses using THz nano-gap metamaterials
2. Terahertz biosensing metamaterial absorber for virus detection based on spoof surface plasmon polaritons. International journal of RF and microwave, doi 10.1002/mmce.21448

1-35. (canceled)

36. A method for prompt virus infection carriers detection/screening using THz spectroscopy, comprising:
a) providing a micro/nano-antennas array implemented as an antenna chip of predetermined shape and size, that has the maximum aspect ratio of the capacitor gap being sensitive to both P and S polarization, said array consisting of a plurality of printed micro-antenna elements, each of which having an equivalent inductor L of printed inductors and an equivalent capacitor C defined by gaps between printed contacts the length of the capacitor and the dielectric constant of a filler being between said printed contacts, thereby determining a resonant frequency of said antenna element, said gaps are formed essentially along the cross diagonals of said each antenna element, thereby obtaining maximal aspect-ratio between the length of said capacitor and the gap width, that maximizes and sharpen the resonance effect of said each micro-antenna element;
b) altering said dielectric constant of said filler by applying material containing samples of viruses/exhaled biological ingredients to be detected, into said gaps, thereby altering said resonance frequency;
c) detecting shifts in said resonance frequency induced by the presence of said viruses/biological ingredients that are exhaled in said gaps by scanning said samples using a spectrometer; and
d) associating different shifts in said resonance frequency with different types of viruses/biological ingredients,
wherein the size of said array is matched to the beam size of said spectrometer, such that the entire radiation collimated beam will be captured by said antennas array, thereby maximizing the signal to noise ratio and the dynamic range.

37. The method according to claim 36, further comprising associating corresponding shifts with healthy individuals and confirmed sick individuals, to further increase the probability of detection and minimize the false negative and false positive indications.

38. The method according to claim 37, further comprising applying machine learning analysis after collecting a large amount of THz spectra, and for wide frequency span by scanning using THz radiation in transmission or reflection mode spectrometers, for further increasing the possibility of detection.

39. The method according to claim 36, wherein the combination of the inductance and the capacitance in the antenna element is in the range of pico-Henry and Femto-Farad, respectively.

40. The method according to claim 36, wherein the substrate has a low doping level up to intrinsic semiconductor or insulator, thereby minimizing the free carrier absorption of the THz radiation, mainly in transmission mode.

41. The method according to claim 36, wherein THz spectroscopy is done for dual polarizations P and S or for a single polarization spectrometer.

42. The method according to claim 36, wherein the antenna chip is inserted inside a plastic capsule that is part of a breathalyzer where the geometrical shape, plastic thickness and air gap are designed in accordance with the chip thickness and the nano-antennas characteristics thereby minimizing the internal reflection and standing waves.

43. The method according to claim 36, wherein spectroscopy is done manually or by an automatic system that has a synchronous loader and into the spectrometer, where a loader loads tagged chips mounted inside the capsule into the spectrometer, which frequency scans each chip for few tens of seconds analyze the frequency shift and provides during a diagnostic mode accurate indications whether a person is infected—or during a screening mode, whether a person is not infected.

44. The method according to claim 36, wherein the structure of each cell is a diagonal type micro-antenna cell, based on different capacitors that are in multiple orientations, to be responsive to different polarization P and S.

45. The method according to claim 36, wherein samples are collected using a breathalyzer with a mouthpiece configuration that is adapted to generate a fine mist after the breath test on the chip surface, for allowing said mist to dry out in few second after the breath test.

46. The method according to claim 36, wherein a reference chip without the viruses/biological ingredients is measured in advance and each spectrum is compared with that reference chip, in order to analyze the spectral shift.

47. The method according to claim 36, wherein the chip with the antennas array is mounted in a cylindrical capsule in a fixed position, preferably at the center, said capsule being at least partially transparent to THz radiation range and being sealed to prevent contamination during a clinical trial.

48. The method according to claim 36, wherein the metal surface of the antennas is with high conductivity, to thereby obtain a high Q-factor and is thicker than the skin depth of the THz radiation for said metal.

49. A system for prompt virus infection carriers detection/screening using THz spectroscopy, comprising:
a) a micro/nano-antennas array implemented as an antenna chip of predetermined shape and size, that has the maximum aspect ratio of the capacitor gap being sensitive to both P and S polarization, said array consisting of a plurality of printed micro-antenna elements, each of which having an equivalent inductor L of printed inductors and an equivalent capacitor C defined by gaps between printed contacts the length of the capacitor and the dielectric constant of a filler being between said printed contacts, to thereby determine a resonant frequency of said antenna element, said gaps are formed essentially along the cross diagonals of said each antenna element, thereby obtaining maximal aspect-ratio between the length of said capacitor and the gap width, that maximizes and sharpen the resonance effect of said each micro-antenna element;
b) at least one capsule for holding the chip with the antennas array in a fixed position, preferably at the center, said at least one capsule being at least partially transparent to THz radiation range;
c) means for applying material containing samples of viruses/exhaled biological ingredients to be detected that are exhaled into said gaps, for altering said dielectric constant of said filler and said resonance frequency;
d) a spectrometer, such as a THz spectrometer, for scanning said samples and detecting shifts in said resonance frequency induced by the presence of said exhaled viruses/biological ingredients; and
e) at least one processor for processing the detected shifts in said resonance frequency and associating different shifts with different types of viruses/biological ingredients,
wherein the size of said array is matched to the beam size of said spectrometer, such that the entire radiation collimated beam will be captured by said antennas array, thereby maximizing the signal to noise ratio and the dynamic range.

50. The system according to claim 49, in which the capsule is sealed to prevent contamination during a clinical trial.

51. The system according to claim 49, in which the at least one processor is further adapted to:
a) associate corresponding shifts with healthy individuals and confirmed sick individuals, to further increase the probability of detection and minimize the false negative and false positive indications.
b) apply machine learning analysis after collecting large amount of THz spectra and for wide frequency span, for further increasing the detection probability.

52. The system according to claim 49, further comprising a breathalyzer containing the antenna chip inserted inside a plastic capsule where the geometrical shape, plastic thickness and air gap are designed in accordance with the chip thickness and the nano-antennas characteristics, thereby minimizing the internal reflection and standing waves.

53. The system according to claim 49, further comprising a synchronous loader for loading tagged chips mounted inside the capsule into the spectrometer, which frequency scans each chip for few tens of seconds, analyzes the frequency shift and provides, during a diagnostic mode, accurate indications whether a person is infected or during a screening mode, whether a person is not infected.

54. The system according to claim 49, in which the breathalyzer further comprises a mouthpiece that is adapted to generate a fine mist after the breath test on the chip surface, for allowing said mist to dry out in few second after the breath test.

55. The system according to claim 14, further comprising a linear or circular magazine for performing multiple tests, inside which a plurality of capsules are mounted, said magazine each time performs a predetermined displacement, in order to advance a single capsule into the spectrometer, to coincide with the beam of said spectrometer.

56. A breathalyzer containing the antenna chip inserted inside a capsule where the geometrical shape, thickness and air gap of said capsule are designed in accordance with the chip thickness and the nano-antennas characteristics, thereby minimizing the internal reflection and standing waves.
